(19) United States
(12) Patent Application Publication
(10) Pub. No.: US 2024/0268650 A1
(43) Pub. Date: Aug. 15, 2024

(54) ENDOSCOPE SYSTEM AND METHOD OF OPERATING THE SAME

(71) Applicant: FUJIFILM Corporation, Tokyo (JP)

(72) Inventor: Takanori DEMURA, Kanagawa (JP)

(73) Assignee: FUJIFILM Corporation, Tokyo (JP)

(21) Appl. No.: 18/441,984

(22) Filed: Feb. 14, 2024

(30) Foreign Application Priority Data
Feb. 14, 2023 (JP) 2023-020686

(51) Int. Cl. A61B 1/06; A61B 1/07

(52) U.S. Cl. CPC A61B 1/0655; A61B 1/063; A61B 1/0638; A61B 1/0684; A61B 1/07

(57) ABSTRACT

A drive current value is set larger than a target drive current value of the light source, the light source emits illumination light with an amount corresponding to the drive current value in a light emission period shorter than a determined exposure period, an amount of received light for each certain period is acquired a plurality of times within the light emission period, a cumulative amount of light that is the amount of light emission for each certain period is calculated based on the amount of received light, the illumination light is turned off for a predetermined period in a case in which a total value of the cumulative amounts of light reaches a target integrated amount of light, a captured image obtained by imaging an observation target is acquired for each light emission period, and one observation image is generated from a plurality of the captured images.

### CROSS-REFERENCE TO RELATED APPLICATIONS

This application claims priority under 35 U.S.C § 119(a) to Japanese Patent Application No. 2023-020686 filed on 14 Feb. 2023. The above application is hereby expressly incorporated by reference, in its entirety, into the present application.

### BACKGROUND OF THE INVENTION

### 1. Field of the Invention

The present invention relates to an endoscope system and a method of operating the same.

### 2. Description of the Related Art

In recent years, in a medical field, diagnosis and the like using an endoscope system have been widely performed, and a semiconductor light source such as a laser diode (LD) or a light emitting diode (LED) that emits light of a specific color, which is useful for energy saving, long life, and narrow-band light observation, has been used as a light source of a light source device for an endoscope included in the endoscope system.

For the diagnosis using the endoscope system, various observation methods have been proposed for an observation target. For example, there are two types of observation modes: a single-frame observation mode in which an observation image is generated using one or a plurality of captured images obtained from one type of imaging frame; and a multi-frame observation mode in which one observation image is generated using a plurality of captured images obtained from a plurality of types of imaging frames. The multi-frame observation mode is an observation mode in which imaging of the observation target is performed for each illumination light by controlling turning-on and turning-off (light emission timing) of a plurality of light sources, and an example thereof is an endoscope system disclosed in JP2020-36780A (corresponding to US2020/069163A1).

In the endoscope system disclosed in JP2020-36780A, there is a technique of making an image signal ratio between images acquired for each illumination light constant in the multi-frame observation mode. In a predetermined certain period, a difference between an amount of light detected by a photodetector and a target amount of light is calculated, and a turning-off timing of the illumination light is adjusted.

### SUMMARY OF THE INVENTION

In a control of the amount of light disclosed in JP2020-36780A, an integrated amount of light within an exposure time is adjusted by adjusting the turning-off timing, but a control of the amount of light only in the exposure time does not prevent deterioration of image quality caused by a variation in a drive current for each device. In JP2020-36780A, a variation in a drive current of the semiconductor light source cannot be absorbed, and a cumulative amount of light varies. In a case in which a variation in the cumulative amount of light increases, an image captured by a sensor for the same subject changes, resulting in deterioration of image quality in a case in which a multi-frame observation image is created. The light output in a case in which the amount of light is low has a large influence on the cumulative amount of light.

An object of the present invention is to provide an endoscope system and a method of operating the same with which illumination light corresponding to a target amount of light is emitted even in a case in which there is a variation in a drive current, and highly accurate and stable imaging is performed.

According to one aspect of the present invention, there is provided an endoscope system comprising: at least one light source having a semiconductor light emitting element; a photodetector that receives a part of light from the light source and detects an amount of received light; and a processor, in which the processor determines a drive current value that is a value larger than a target drive current value of the light source set in advance, causes the light source to emit illumination light with an amount of light emission corresponding to the drive current value in a light emission period of the light source that is shorter than a determined exposure period, acquires the amount of received light from the photodetector for each certain period a plurality of times within the light emission period, calculates a cumulative amount of light that is the amount of light emission for each certain period, based on the amount of received light, turns off the illumination light for a predetermined period in a case in which a total value of the cumulative amounts of light reaches a target integrated amount of light set in advance, acquires a captured image obtained by imaging an observation target during the light emission period, and generates one observation image from a plurality of the captured images acquired for each light emission period.

It is preferable to emit the illumination light at the drive current value increased from the target drive current value by a constant magnification.

It is preferable to emit the illumination light at the drive current value increased from the target drive current value by a fixed value.

It is preferable to emit the illumination light at the drive current value that is increased by a first magnification in a case in which the target drive current value is equal to or larger than a threshold value and is increased by a second magnification higher than the first magnification in a case in which the target drive current value is less than the threshold value.

It is preferable to emit the illumination light at the drive current value that is increased by a constant magnification in a case in which the target drive current value is equal to or larger than a threshold value and is increased by a fixed value in a case in which the target drive current value is less than the threshold value.

It is preferable to set a light emission timing of the illumination light in accordance with an end of the determined exposure period.

It is preferable that the certain period is 0.5 μs or more and 100 μs or less.

It is preferable to acquire a variation between a set value of the target drive current value and an actual measurement value of a drive current transmitted to the light source, and to set a lower limit value of the drive current value based on the variation.

It is preferable to determine a method of increasing the drive current value with respect to the target drive current value according to the lower limit value.

It is preferable to control exposure of light incident into an imaging sensor through a shutter control in a state where the light source continues to emit light, instead of light emission and turning-off of the light source.

It is preferable that the semiconductor light emitting element is a laser diode or a light emitting diode.

It is preferable to emit first illumination light and second illumination light, which are light beams having different wavelengths, according to the respective drive current values.

According to another aspect of the present invention, there is provided a method of operating an endoscope system including at least one light source having a semiconductor light emitting element and a photodetector that receives a part of light from the light source and detects an amount of received light, the method comprising: a step of determining a drive current value that is a value larger than a target drive current value of the light source set in advance; a step of causing the light source to emit illumination light with an amount of light emission corresponding to the drive current value in a light emission period of the light source that is shorter than a determined exposure period; a step of acquiring the amount of received light from the photodetector for each certain period a plurality of times within the light emission period; a step of calculating a cumulative amount of light that is the amount of light emission for each certain period, based on the amount of received light; a step of turning off the illumination light for a predetermined period in a case in which a total value of the cumulative amounts of light reaches a target integrated amount of light set in advance; a step of acquiring a captured image obtained by imaging an observation target during the light emission period; and a step of generating one observation image from a plurality of the captured images acquired for each light emission period.

According to the present invention, it is possible to emit illumination light corresponding to a target amount of light even in a case in which there is a variation in a drive current, and to realize highly accurate and stable imaging.

### BRIEF DESCRIPTION OF THE DRAWINGS

FIG. 1 is a schematic view of an endoscope system.

FIG. 2 is a block diagram showing a function of the endoscope system.

FIG. 3 is an explanatory diagram showing that a photodetector receives a part of light emitted from a light source.

FIG. 4 is an explanatory diagram of a feedback control in a light source device.

(A) of FIG. 5 is an explanatory diagram showing a relationship between a target amount of light, and an exposure period and an amount of illumination light, and (B) of FIG. 5 is an explanatory diagram showing a relationship between a drive current value and the amount of illumination light.

(A) of FIG. 6 is an explanatory diagram showing a relationship between a target amount of light in a case in which a global exposure period is considered, and an exposure period and an amount of illumination light, and (B) of FIG. 6 is an explanatory diagram showing a relationship between a drive current value and the amount of illumination light in a case in which the global exposure period is considered.

FIG. 7 is an explanatory diagram of a variation in an amount of light emission caused by a variation in a drive current value.

FIG. 8 is an explanatory diagram showing that a target integrated amount of light is emitted with an amount of light emission larger than a target amount of light emission.

FIG. 9 is an explanatory diagram of a cumulative amount of light acquired by accumulating an amount of light emission for each sampling period.

FIG. 10 is an explanatory diagram of a configuration of a light source device including two types of light sources.

FIG. 11 is an explanatory diagram of a feedback control in a light source device including two types of light sources.

FIG. 12 is an explanatory diagram of a light emission pattern in which multi-frame light emission is performed with a target amount of light emission.

FIG. 13 is an explanatory diagram of a light emission pattern in which an amount of light emission is increased from a target amount of light emission by a constant magnification.

FIG. 14 is an explanatory diagram of a light emission pattern in which an amount of light emission is increased from a target amount of light emission by a fixed value.

FIG. 15 is an explanatory diagram of a light emission pattern in which a rate of increase is changed according to a target amount of light emission.

FIG. 16 is an explanatory diagram of a light emission pattern in which an amount of light emission is increased from a target amount of light emission by a constant magnification and a fixed value.

FIG. 17 is an explanatory diagram of a light emission pattern in which an amount of light is increased in accordance with an end timing of the global exposure period.

FIG. 18 is a flowchart showing a series of flows of a control of an amount of light according to the present invention.

FIG. 19 is an explanatory diagram of a configuration of a light source unit including four types of light sources.

FIG. 20 is an explanatory diagram of a feedback control in a light source device including four types of light sources.

FIG. 21 is an explanatory diagram of a wavelength range of illumination light from four types of light sources.

### DESCRIPTION OF THE PREFERRED EMBODIMENTS

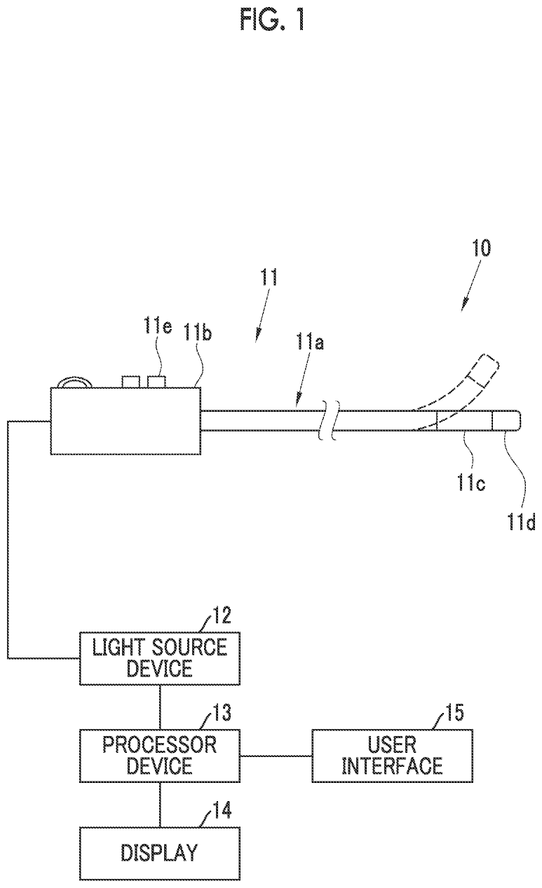

As shown in FIG. 1, an endoscope system 10 according to an embodiment of the present invention includes an endoscope 11, a light source device 12, a processor device 13, a display 14, and a user interface (UI) 15. The endoscope 11 is optically connected to the light source device 12 and electrically connected to the processor device 13. The light source device 12 supplies illumination light to the endoscope 11.

The endoscope 11 emits illumination light and images an observation target to acquire an endoscopic image. The endoscope 11 has an insertion part 11a that is to be inserted into a living body (inside an object to be examined) having the observation target, and an operation part 11b that is provided at a base end portion of the insertion part 11a. A bendable part 11c and a distal end part 11d are provided on a distal end side of the insertion part 11a. The bendable part 11c is operated by the operation part 11b to be bent in a desired direction. The distal end part 11d irradiates the observation target with illumination light and receives reflected light from the observation target to image the observation target. The operation part 11b is provided with a mode selector switch 11e that is used for an operation for switching a mode.

The processor device 13 is electrically connected to the display 14 and the user interface 15. The processor device 13 receives an image signal from the endoscope 11, and performs various kinds of processing based on the image signal. An external recording unit (not shown), which records an image, image information, and the like, may be connected to the processor device 13. The display 14 outputs and displays a captured image of the observation target, image information, and the like, which have been image-processed by the processor device 13. The user interface 15 includes a keyboard, a mouse, a touch pad, a microphone, a foot pedal, and the like, and has a function of receiving an input operation such as function setting.

The endoscope system 10 can execute normal observation for observing an observation site and special observation for enhancing and observing a specific structure in the observation site. In the special observation, a plurality of frames including a frame illuminated with illumination light of a specific wavelength to enhance a specific structure are imaged to generate one observation image. The generated observation image is displayed on the display 14.

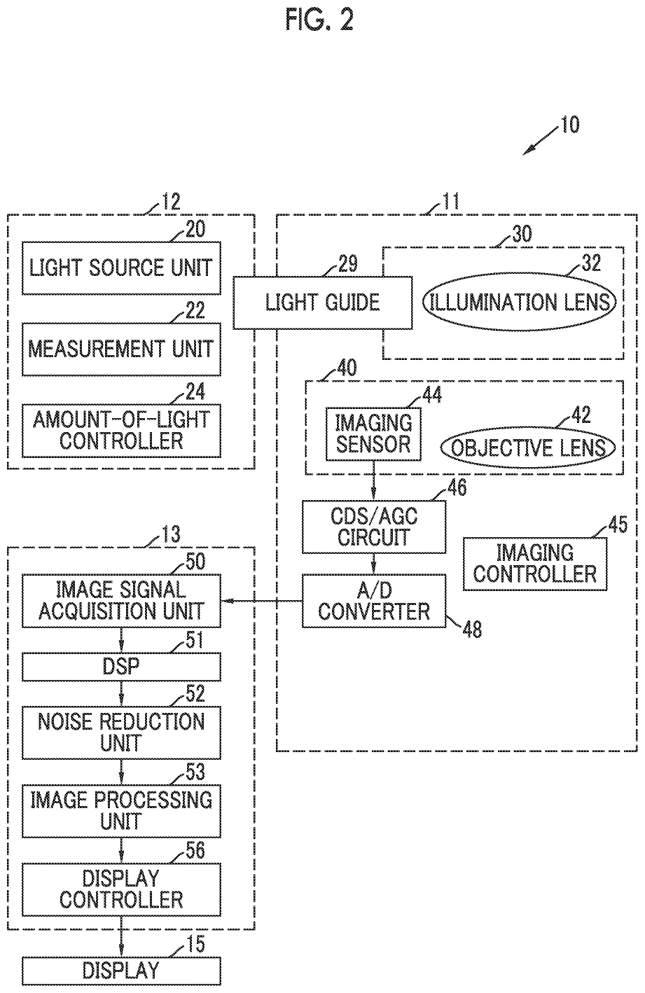

As shown in FIG. 2, in the endoscope system 10, the light source device 12 propagates emitted illumination light to the endoscope 11 via a light guide 29, the endoscope 11 transmits an image signal imaged using illumination light to the processor device 13, and the processor device 13 generates an image to be displayed on the display 14.

The light source device 12 comprises a light source unit 20 that emits a plurality of illumination light beams having different main wavelengths and detects an amount of the emitted illumination light, a measurement unit 22 that measures a cumulative amount of the illumination light in a predetermined period and acquires a difference between the cumulative amount of light and a target amount of light, and an amount-of-light controller 24 that generates a drive current (drive signal) for controlling a light emission timing and an amount of light emission of the light source unit 20 and supplies the drive current to the light source unit 20 to emit light. The target amount of light is a target value of an amount of emitted illumination light.

The functions of the measurement unit 22 and the amount-of-light controller 24 are realized by a light source control processor (not shown) provided in the light source unit 20, and the illumination light emitted from the light source unit 20 is controlled. In a case in which the light source device 12 and the processor device 13 are electrically connected, the function of the light source control processor may be realized by a central controller instead of the light source control processor.

The light emitted from the light source unit 20 is incident into the light guide 29. The light guide 29 is built in the endoscope 11 and a universal cord (a cord connecting the endoscope 11, the light source device 12, and the processor device 13). The light guide 29 propagates the light from the light source unit 20 to the distal end part 11d of the endoscope 11.

An illumination optical system 30 and an imaging optical system 40 are provided at the distal end part 11d of the endoscope 11. The illumination optical system 30 has an illumination lens 32, and the illumination light propagated by the light guide 29 is applied to the observation target via the illumination lens 32. The imaging optical system 40 includes an objective lens 42 and an imaging sensor 44. Reflected light of the illumination light returning from the observation target irradiated with the illumination light is incident into the imaging sensor 44 via the objective lens 42. Thereby, an image of the observation target is formed on the imaging sensor 44 which is a color imaging sensor.

An imaging controller 45 drives and controls the imaging sensor 44 according to the mode selector switch 11e, an instruction input from the user interface 15 via the processor device 13, and a signal from the amount-of-light controller 24 to perform mode switching of the observation mode and control the imaging in each mode. In the control of the imaging, adjustment of an exposure period through setting of a shutter speed of an electronic shutter (not shown) of the imaging sensor 44 is performed. In a case in which the mode switching is performed, the illumination light emitted via the amount-of-light controller 24 is switched.

In a case of a single-frame observation mode, since a length of an imaging frame is constant, the imaging sensor 44 is controlled to alternately repeat an accumulation period and a readout period every certain time, for example, every 1/60 seconds. The shutter speed of the electronic shutter may be changed to adjust the length of the imaging frame.

As the imaging sensor 44, a photoelectric conversion element such as a charge coupled device (CCD) sensor or a complementary metal-oxide semiconductor (CMOS) sensor is used. The imaging sensor 44 performs, for example, an accumulation operation of performing photoelectric conversion of received light and accumulating signal charges corresponding to the amount of received light for each pixel and a readout operation of reading out the accumulated signal charges, within an acquisition period of one frame. The signal charge for each pixel read out from the imaging sensor 44 is converted into a voltage signal and is input to a correlated double sampling/automatic gain control (CDS/AGC) circuit 46. The light source device 12 generates illumination light in accordance with a timing of the accumulation operation of the imaging sensor 44 and causes the illumination light to be incident into the light guide 29.

Each pixel of the imaging sensor 44 is provided with any of a blue pixel (B pixel) having a blue (B) color filter, a green pixel (G pixel) having a green (G) color filter, or a red pixel (R pixel) having a red (R) color filter. For example, the imaging sensor 44 is preferably a color imaging sensor of a Bayer array in which a ratio of the number of pixels of the B pixels, the G pixels, and the R pixels is 1:2:1.

The B color filter mainly transmits light in a blue band, specifically, light of which a wavelength range is 380 to 560 nm (blue transmission range). A peak wavelength at which a transmittance is maximized exists around 460 to 470 nm. The G color filter mainly transmits light in a green band, specifically, light of which a wavelength range is 450 to 630 nm (green transmission range). The R color filter mainly transmits light in a red band, specifically, light of which a wavelength range is 580 to 760 nm (red transmission range).

In addition, a complementary color imaging sensor comprising complementary color filters corresponding to cyan (C), magenta (M), yellow (Y), and green (G) may be used instead of the primary color imaging sensor 44. In a case in which the complementary color imaging sensor is used, image signals corresponding to four colors of C, M, Y, and G are output. Therefore, in a case in which the image signals corresponding to four colors of C, M, Y, and G are converted into image signals corresponding to three colors of R, G, and B by complementary color-primary color conversion, image signals corresponding to the same respective colors of R, G, and B as those of the imaging sensor 44 can be obtained.

The CDS/AGC circuit 46 performs correlated double sampling (CDS) or automatic gain control (AGC) on the analog image signals obtained from the imaging sensor 44. The image signal that has passed through the CDS/AGC circuit 46 is converted into a digital image signal by an analog/digital (A/D) converter 48. The digital image signal after the A/D conversion is input to the processor device 13.

In the processor device 13, a program related to each processing is incorporated in a program memory (not shown). In a case in which a central controller (not shown) configured by a processor executes the program in the program memory, functions of an image signal acquisition unit 50, a digital signal processor (DSP) 51, a noise reduction unit 52, an image processing unit 53, and a display controller 56 are realized.

The image signal acquisition unit 50 receives an image signal input from the endoscope 11, which is subjected to a drive control by the imaging controller 45, and transmits the received image signal to the DSP 51. The display controller 56 transmits an image signal of an image to be displayed, which is acquired from the image processing unit 53, to the display 14.

The DSP 51 performs various kinds of signal processing, such as defect correction processing, offset processing, gain correction processing, linear matrix processing, gamma conversion processing, demosaicing, and YC conversion processing, on the received image signal. In the defect correction processing, a signal of a defective pixel of the imaging sensor 44 is corrected. In the offset processing, a dark current component is removed from the image signal that has passed through the defect correction processing, and an accurate zero level is set. In the gain correction processing, a signal level of each image signal is adjusted by multiplying the image signal of each color after the offset processing by a specific gain. The image signal of each color after the gain correction processing is subjected to the linear matrix processing for enhancing color reproducibility.

After that, brightness and chroma saturation of each image signal are adjusted by the gamma conversion processing. The image signal after the linear matrix processing is subjected to the demosaicing (also referred to as isotropic processing or synchronization processing), and a signal of a color missing from each pixel is generated by interpolation. By the demosaicing, all pixels have signals of respective colors of R, G, and B. The DSP 51 performs the YC conversion processing on each image signal after the demosaicing, and outputs a brightness signal Y, a color difference signal Cb, and a color difference signal Cr to the noise reduction unit 52.

The noise reduction unit 52 performs noise reduction processing by, for example, a moving average method or a median filter method on the image signal that has passed through the demosaicing or the like by the DSP 51. The image signal with reduced noise is input to the image processing unit 53.

The image processing unit 53 switches processing of the image signal from the noise reduction unit 52 according to the set observation mode. In the present embodiment, single-frame image processing is executed in a case in which the observation mode is set to the single-frame observation mode, and multi-frame image processing is executed in a case in which the observation mode is set to the multi-frame observation mode.

In any image processing, the image processing unit 53 further performs color conversion processing such as 3×3 matrix processing, gradation transformation processing, and three-dimensional look up table (LUT) processing on the input image signal for one frame. Then, various kinds of color enhancement processing are performed on the RGB image data that has been subjected to the color conversion processing. Structure enhancement processing such as spatial frequency enhancement is performed on the RGB image data that has been subjected to the color enhancement processing. In the single-frame image processing, the RGB image data that has passed through the structure enhancement processing is input to the display controller 56 as a normal light observation image. In the multi-frame image processing, one special light observation image is generated based on the illumination light from the plurality of RGB image data that has passed through the structure enhancement processing. The generated special light observation image is input to the display controller 56.

The display controller 56 sequentially acquires the normal light observation images or the special light observation images from the image processing unit 53, and converts the obtained light observation images into video signals that enable full-color display on the display 14. The converted video signal is output to and displayed on the display 14. Accordingly, a doctor or the like can observe the observation target by using a still image or a video image of the observation image.

As described above, the endoscope system 10 according to the present embodiment has two types of observation modes: the single-frame observation mode used for normal observation in which the illumination light under observation is continuously turned on; and the multi-frame observation mode used for special observation such as vascular enhancement observation in which an operation of turning off and on at least two types of illumination light beams having different wavelengths in a short time is repeated. The control of the amount-of-light controller 24 is different for each mode, and the turning-on or turning-off of each light source and the amount of light emission in a case in which the light source is turned on are independently controlled. In addition, it is preferable that opening and closing of the shutter is controlled in synchronization with the imaging frame.

In the single-frame observation mode, a normal observation image suitable for observing the entire observation site is generated as a display image. In the normal observation, the light is continuously turned on during the observation, and the exposure period is adjusted by the opening and closing of the shutter or the like.

In the multi-frame observation mode, strobe light emission is performed in which illumination light beams having different wavelengths are turned off and turned on in a short time. In the multi-frame observation mode, in order to obtain a high-quality observation image from a plurality of captured images using illumination light beams having different wavelengths, an exposure time is adjusted according to the illumination light, a variation in the amount of light in each frame due to a variation in a drive current for each device is suppressed, and an image signal ratio between frames is made constant. The smaller the amount of emitted illumination light, the larger the influence of the variation.

The amount-of-light controller 24 generates a control signal for controlling the light emission timing and the amount of light emission of the light source unit 20, and supplies a drive current (drive signal) corresponding to the control signal to each light source to emit light. The amount-of-light controller 24 compares a light amount measurement signal acquired from the measurement unit 22 with a target integrated amount of light and adjusts the drive current based on a comparison result.

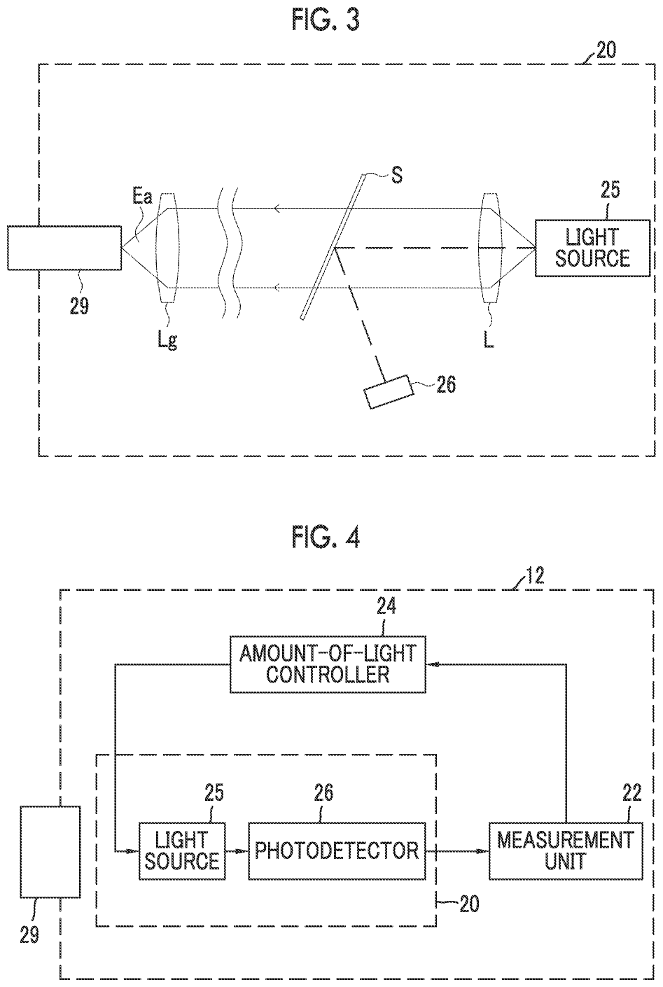

As shown in FIG. 3, in the light source unit 20, the photodetector 26 corresponding to the light source 25 acquires a light amount measurement signal of an amount of light emitted from the light source 25. A lens L, a beam splitter S, and the photodetector 26 are provided in the vicinity of any light source 25. The lens L adjusts light emitted from the light source 25 into parallel light, the beam splitter S reflects a part of the parallel light at a predetermined rate, and the photodetector 26 receives the light reflected by the beam splitter S and detects the light as an amount of received light. The parallel light transmitted through the beam splitter S is incident into the light guide 29 as an incidence ray Ea via a lens Lg that narrows the parallel light. The amount of light emitted from each light source 25, which will be described below, indicates the amount of light that is transmitted through the beam splitter S without being reflected by the beam splitter S and is incident into the light guide 29. In a case in which light emission is switched by one light source to emit different illumination light beams, it is necessary to understand a wavelength range reflected by the beam splitter S and a reflectivity thereof.

The light source 25 emits light according to the drive current generated by the amount-of-light controller 24. A magnitude of the amount of light in light emission is determined based on the drive current value. The photodetector 26 is configured of, for example, a photodiode (PD), in which a current corresponding to the amount of light received by the PD is converted into a voltage and converted into a digital value such as 16 bits by an analog to digital converter (ADC). Information on the amount of received light detected by the photodetector 26 is transmitted to the measurement unit 22.

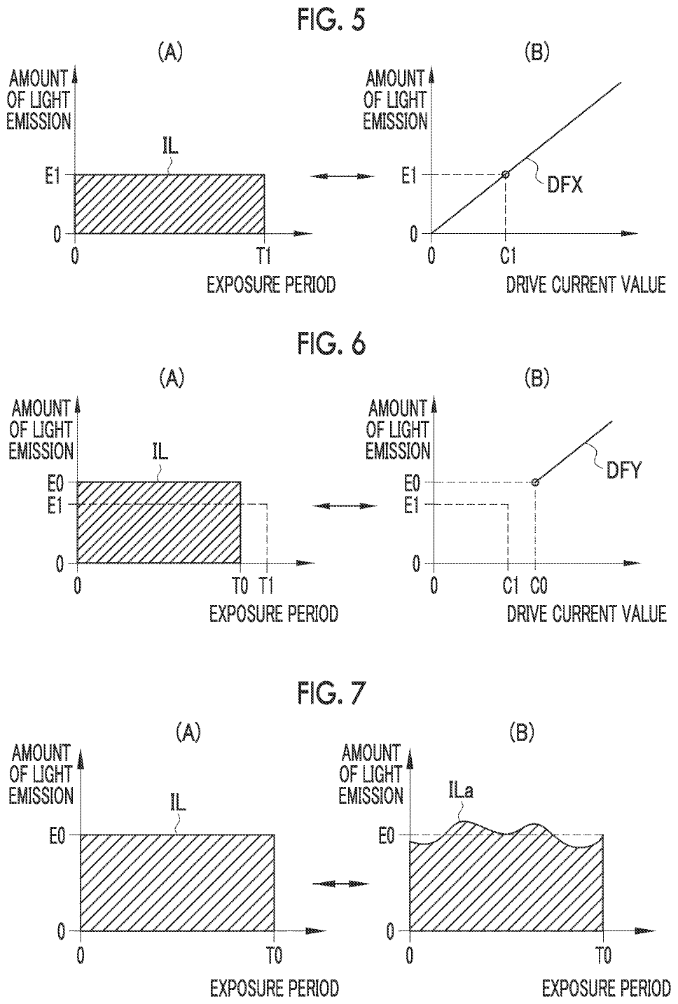

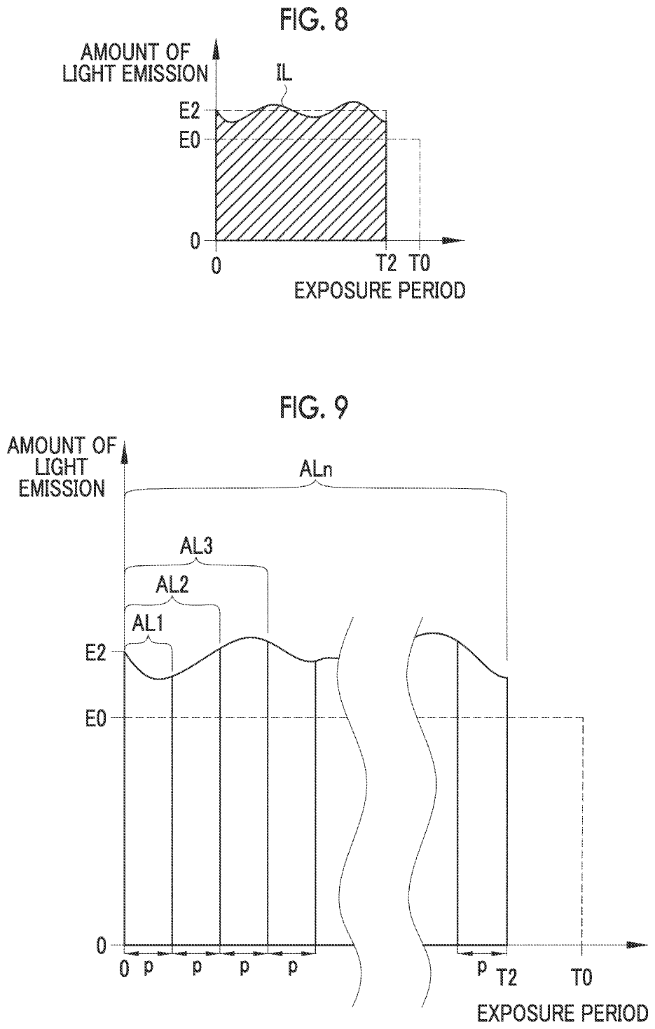

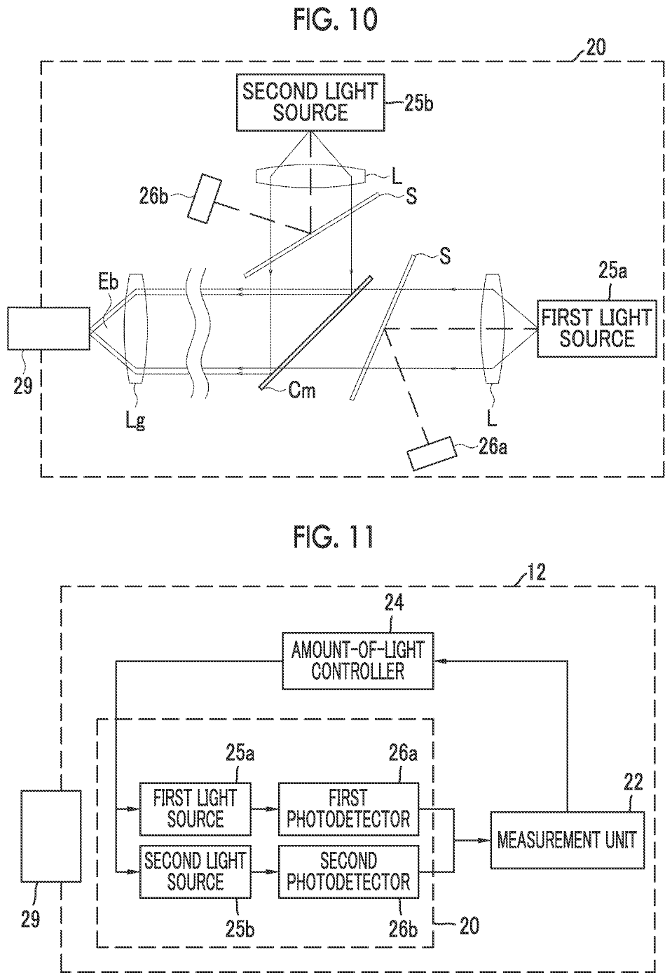

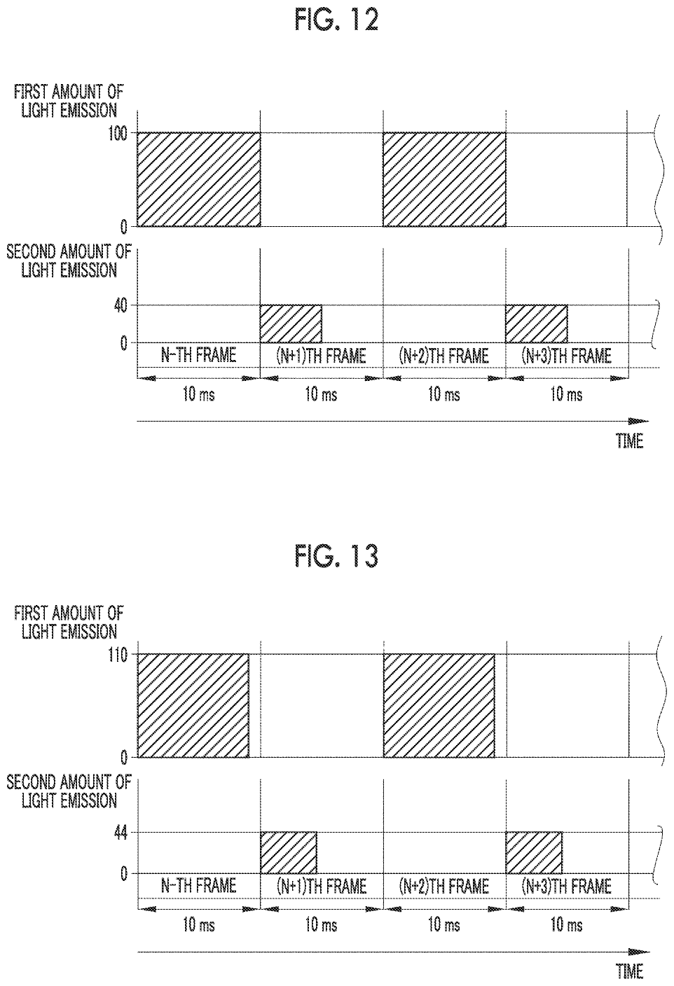

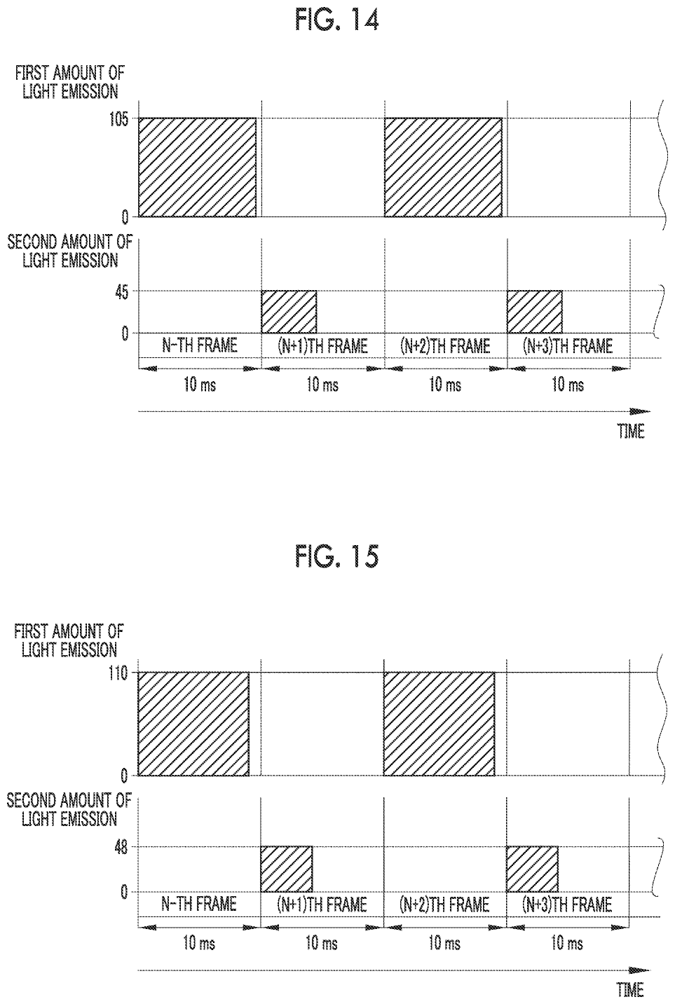

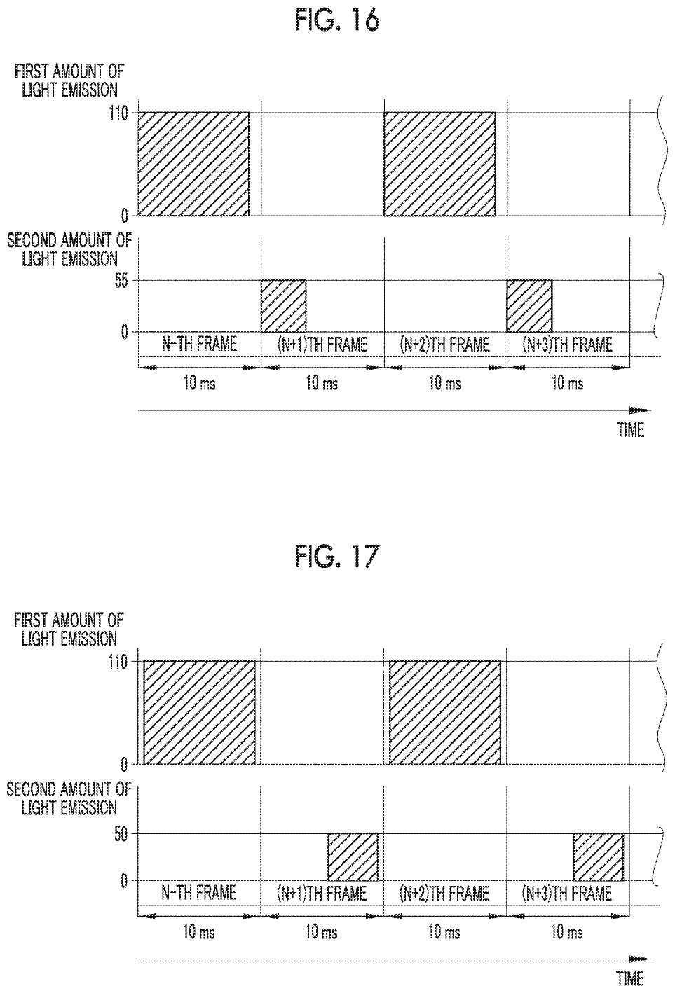

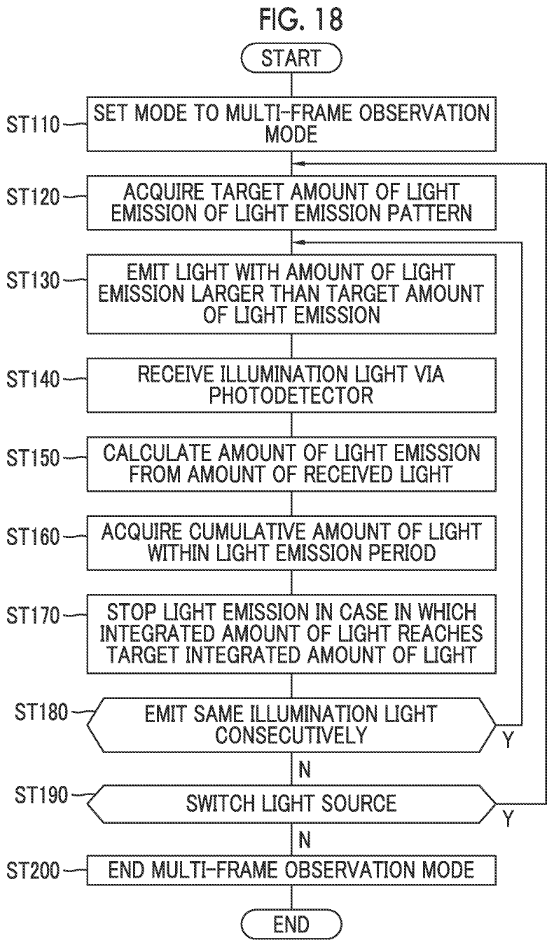

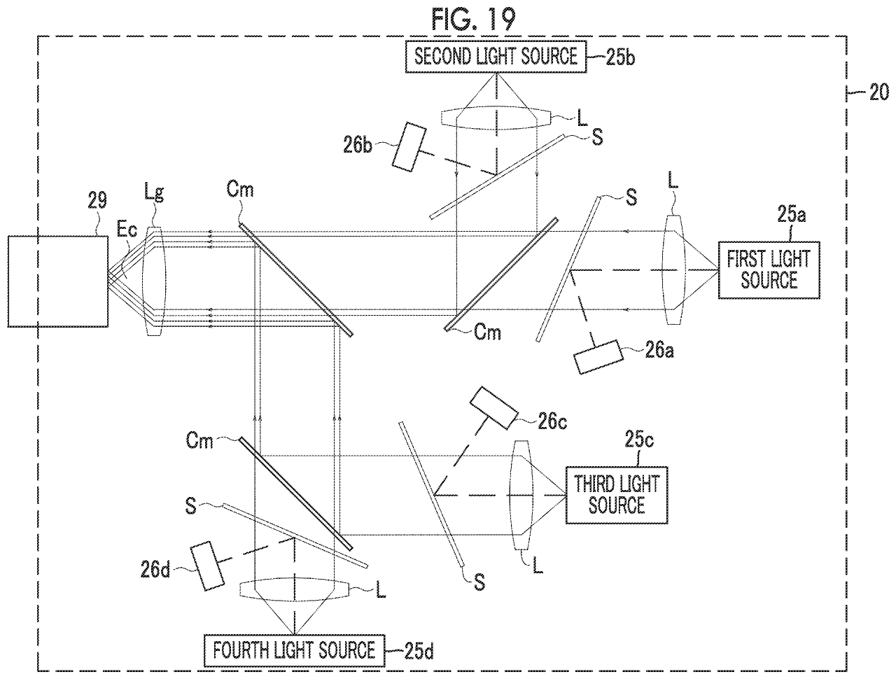

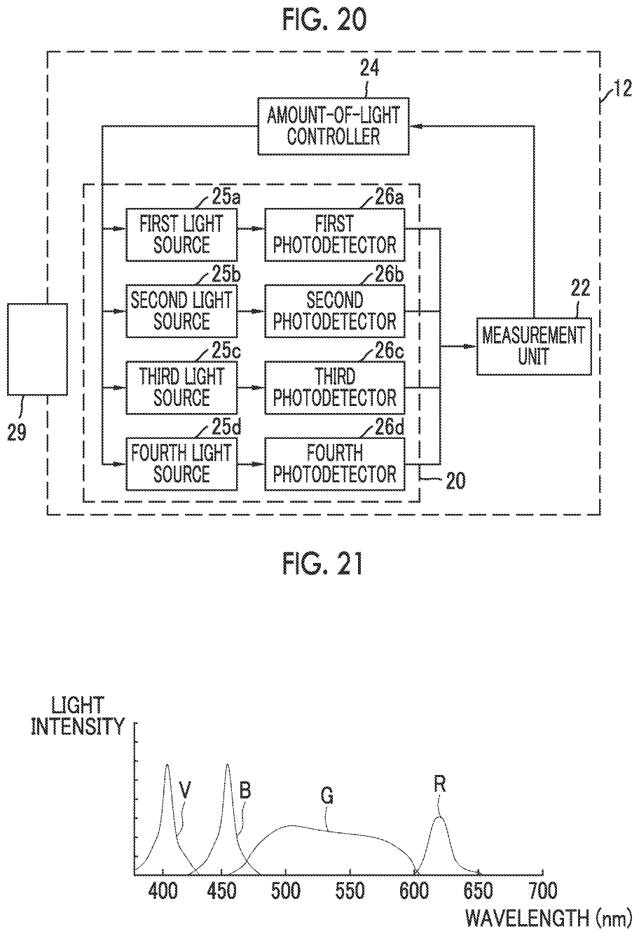

As shown in FIG. 4, in the light source device 12, a feedback control in which the amount-of-light controller 24 adjusts the drive current transmitted to the light source unit 20 is executed to perform a control of the amount of light. A target amount of light emission or the target integrated amount of light is stored in the amount-of-light controller 24 or is input from the processor device 13. The photodetector 26 receives a part of the emission light emitted from the light source 25, which is a laser diode or a light emitting diode, detects the light as an amount of received light, and transmits the light to the measurement unit 22. The measurement unit 22 measures information on the amount of light emission of each light source 25 detected for each certain period from the acquired light amount measurement signal. The amount-of-light controller 24 performs the control of the amount of light based on the amount of light emission.

The light source unit 20 in the light source device 12 comprises a cooling member such as a heat sink that cools a light emitting element of each light source. It is preferable that the light source device 12 comprises a drive current measuring device (not shown) that measures the drive current transmitted to the light source 25 from the amount-of-light controller 24, and that a set value and an actual measurement value of the drive current value are acquired. Accordingly, a difference between the set value and the actual measurement value can be detected as the variation in the drive current value.

The light emitted from the light source 25 of FIGS. 3 and 4 is incident into the light guide 29 and is used for illumination and detection of the amount of received light. In a case in which a plurality of light beams are made incident into the light guide 29 by using a plurality of the light sources 25, the light beams are reflected by using a multiplexing member to be described below. The multiplexing member has a property of reflecting light having a specific wavelength and transmitting light having other wavelengths.

In light emission in one frame, an integrated amount of light, which is a total value of the amounts of light emitted during an exposure period, can be calculated as an amount of light exposed by the imaging sensor 44. A control signal for generating a drive current during the exposure period is set to a constant value, and the light output is maintained at the same value. Since the light emission of each frame is controlled to be stopped in a case in which the integrated amount of light reaches a target integrated amount of light IL that is an integrated amount of light required for the imaging, which is set in advance according to a light emission pattern, the imaging can be performed with constant brightness.

As shown in (A) of FIG. 5, in a case in which light emission of an amount of light emission E1 is performed in an exposure period T1 in light emission in one frame in which light emission of the target integrated amount of light IL is performed, a value of E1×T1 becomes the target integrated amount of light IL, which is an amount of light applied to the imaging sensor 44. The amount of light emission indicates a magnitude of light output emitted per unit time. As shown in (B) of FIG. 5, in a case in which the light source 25 emits light with the amount of light emission E1, a drive current having a drive current value C1 is transmitted from the amount-of-light controller 24 to the light source 25, so that the light source 25 emits light with the amount of light emission E1 corresponding to the drive current. The amount of light emission and the drive current value are in a proportional relationship as shown by a definition line DFX.

Note that, in the imaging sensor 44 of the endoscope 11 that performs imaging using the strobe light emission, there exists a global exposure period in which an allowable period during which all effective pixels are commonly exposed to the same light, that is, a period during which continuous exposure can be performed is determined. In a case in which the imaging sensor 44 is exposed beyond a global exposure period T0, gradation or color shift occurs in a captured image, so that light emission in one frame is performed in a period not exceeding the global exposure period. The exposure may be controlled by a shutter control of the imaging sensor 44 to switch between the turning-on and turning-off in a state where the light source unit 20 continues to emit light, instead of turning-off and turning-on of the light source. In a case in which the light source is turned off, the turning-off state is continued for a predetermined period.

In a case in which the drive current value is small, the value of the amount of light emission is also small, so that a period until the integrated amount of light reaches the target integrated amount of light is lengthened. Therefore, in a case in which the integrated amount of light by the light emission reaches the target integrated amount of light in a time shorter than a determined period such as the global exposure period, a lower limit value of the light emission amount is set. Since the amount of light emission corresponds to the drive current value, the lower limit value of the amount of light emission is determined by setting a lower limit value of the drive current value transmitted to the light source by the amount-of-light controller 24. It is preferable to set a lower limit of a drive current value C transmitted to the light source unit 20.

As shown in (A) of FIG. 6, in a case where a value of an amount of light emission E required to achieve the target integrated amount of light IL in the global exposure period T0 is a target amount of light emission E0 in light emission in one frame, a value of E0×T0 becomes the target integrated amount of light IL. In a case in which the amount of light emission E1 in (A) of FIG. 5 is a value smaller than the target amount of light emission E0, the exposure period T1 for reaching the target integrated amount of light IL is longer than the global exposure period T0. Therefore, in order to cause the integrated amount of light to reach the target integrated amount of light IL in light emission for a period shorter than the global exposure period T0, it is preferable set provide the lowest value such that the amount of light emission is always emitted at a value equal to or higher than a certain value. As shown in (B) FIG. 6, the amount of light emission increases or decreases according to the drive current value C, so that it is linked with the lowest value of the amount of light emission E and the drive current value C, and possible values of the amount of light emission E and the drive current value C in a case in which the lowest value is set are as shown by a definition line DFY.

As shown in (A) of FIG. 7, in a case in which the target integrated amount of light IL is applied to the imaging sensor 44 in the global exposure period T0 in each frame, a drive current is transmitted from the amount-of-light controller 24 so that the light source unit 20 emits light with the target amount of light emission E0. Note that, a variation in the drive current occur in a case in which the drive current corresponding to the control signal is transmitted to the light source unit 20. As shown in (B) of FIG. 7, in a case in which there is a variation in the drive current, an actual amount of light emission exceeds or falls below the target amount of light emission E0 even in a case in which the amount-of-light controller 24 performs a control to achieve the target amount of light emission E0. Therefore, an integrated amount of light ILa obtained in the global exposure period T0 can be a value different from the target integrated amount of light IL. The variation in the integrated amount of light leads to an imaging variation in which a ratio of brightness or the like between the captured images is unstable, and, in a case in which one observation image is acquired from a plurality of captured images, the accuracy of synthesis based on a light amount ratio deteriorates.

In order to acquire a frame in which the target integrated amount of light IL is achieved within the global exposure period T0 without being affected by the variation in the drive current, there is a method of controlling the amount of light such that the light emission is stopped in a case in which the integrated amount of light reaches the target integrated amount of light IL by performing the exposure in an exposure period shorter than the global exposure period T0 with an amount of light emission larger than the target amount of light emission E0. Using the photodetector 26 corresponding to the light source 25, a signal of the amount of light of each frame is monitored based on the detected PD light-receiving value, the amount of received light for each certain period is measured as the cumulative amount of light, a timing at which the integrated amount of light reaches the target integrated amount of light IL is detected, and the stop of the light emission is controlled. In addition, the integrated amount of light in one frame is measured using the feedback control by the APC.

As shown in FIG. 8, in order to reliably receive the integrated amount of light with the target integrated amount of light IL within the global exposure period T0, the light source unit 20 emits light with an amount of light emission E2 larger than the target amount of light emission E0. It is preferable that the amount of light emission E2 is determined from a fluctuation width of the variation such that the amount of light emission E2 is not equal to or less than the target amount of light emission E0 even in a case in which a variation in the drive current occurs. By emitting light with the amount of light higher than the target amount of light emission E0, the target integrated amount of light IL can be achieved in a time shorter than the global exposure period T0 even in a case in which the amount of light emission is reduced due to the variation. It is preferable to set a lower limit value of the amount of light emission E2 changed from the target amount of light emission E0 to be a value larger than the maximum value of the variation.

In order to prevent the integrated amount of light from becoming a value larger than the target integrated amount of light IL, the integrated amount of light is measured in real time as the cumulative amount of light, and the light emission is ended at a timing at which the integrated amount of light reaches the target integrated amount of light IL. The amount of light emission in the imaging frame is measured in small increments at a sampling period that is a certain period shorter than the global exposure period, and the acquired cumulative amount of light is measured. For example, with respect to a global exposure period of 10 ms, measurement is performed for each certain period at a sampling period of 1 μs or 4 μs.

As shown in FIG. 9, the amount of light emission is measured for each set sampling period of p seconds, and the light emission is stopped at a time point at which a total value thereof reaches the target integrated amount of light IL. Specifically, the light emission with the amount of light emission E2 set higher than the target amount of light emission E0 is measured for each sampling period of p seconds to obtain a cumulative amount of light ALn obtained by accumulating the amount of light emission. The cumulative amount of light ALn is a cumulative value of the amount of light emission measured n times in the sampling period of p seconds. A cumulative amount of light AL1 is an amount of light emission obtained at n=1, that is, in one sampling period of p seconds, and a cumulative amount of light AL2 is a total amount of light emission obtained at n=2, that is, in two sampling periods of p seconds. Similarly, a cumulative amount of light AL3 is a total amount of light emission obtained in a case in which the sampling period of p seconds has passed three times. A control of stopping the light emission at a time point at which the cumulative amount of light ALn reaches the target integrated amount of light IL is executed by using a field programmable gate array (FPGA).

An exposure period T2 is p×n seconds, which is a total time of n times of the sampling periods of p seconds required until the cumulative amount of light ALn reaches the target integrated amount of light IL. The shorter the sampling period, the more finely the cumulative amount of light ALn can be measured, so that the light emission can be stopped at a timing at which a difference between the cumulative amount of light ALn and the target integrated amount of light IL is small.

The amount-of-light controller 24 drives each light source of the light source unit 20 with a drive current value larger than a target drive current value C0 calculated from the target integrated amount of light IL so that the target integrated amount of light is obtained within the global exposure period. In the control of the integrated amount of light, the amount of light emission is read for each certain period, and, in a case in which the amount of light emission reaches the target amount of light, the light emission in the frame is stopped. For example, the sampling period of p seconds is set to any period between 0.5 μs and 100 μs. In order to emit light with the amount of light emission E2 and obtain the target amount of light within the global exposure period T0, the amount-of-light controller 24 performs a control of transmitting the drive current having a value larger than the target drive current value C0 to the light source unit 20 for each sampling period.

Regarding the variation in the drive current value, the influence on the integrated amount of light is relatively small in a case in which the amount of light is high, but a variation in the light output in the integrated amount of light is large in a case in which the amount of light is low. Therefore, even though the light emission period and the amount of light emission are set to be constant, an integrated amount of light in each frame is different, the integrated amount of light, which is the sum of the cumulative amounts of light, does not match the calculated value, and brightness varies.

In the multi-frame observation mode, in generating an observation image using a plurality of frames (captured images) of different illumination light beams, by controlling a ratio of the integrated amount of light amounts between the emitting light beams to a constant value, a ratio of the amounts of received light between the light beams received by the imaging sensor 44 becomes constant. Therefore, in the plurality of frames, ratios of R pixel values, G pixel values, and B pixel values are the same, and a mutual image signal ratio is constant. Therefore, the amount-of-light controller 24 controls the amount of light in one frame such that the integrated amount of light reaches the target integrated amount of light IL without excess or deficiency, in addition to keeping the exposure period within the global exposure period T0.

In order to increase the amount of light emission, it is preferable to use a plurality of patterns, such as a pattern in which the amount of light emission is changed from the target amount of light emission E0 by a magnification or a pattern in which the amount of light emission is changed by a fixed value. A case in which the light source unit 20 controls the amounts of two types of illumination light beams will be described as an example. In the multi-frame observation, even though an image of the observation target is acquired for each illumination light, a mutual image signal ratio in a plurality of images is made constant.

In the multi-frame observation using two types of illumination light beams, light emission from the light source unit 20 (refer to FIGS. 3 and 4) including one light source 25 that emits illumination light beams having different wavelength ranges, or light emission from the light source unit 20 (refer to FIGS. 10 and 11) including a first light source 25a and a second light source 25b, which are laser diodes or light emitting diodes, emitting illumination light beams having different wavelength ranges by one type each, as described below, is considered.

As shown in FIG. 10, in the light source unit 20, the first light source 25a and the second light source 25b each of which is controlled in the amount of light and the turning-off timing by the drive current generated by the amount-of-light controller 24 are provided with a first photodetector 26a and a second photodetector 26b, respectively. As in the light source 25 shown in FIG. 3, first light emitted by the first light source 25a is partially reflected by the beam splitter S, the reflected light is received by the first photodetector 26a, and the remaining first light is incident into the light guide 29 via the lens Lg. In addition, as for second light emitted by the second light source 25b, as with the first light, the second photodetector 26b receives reflected light, and the remaining second light is also incident into the light guide 29 via the lens Lg, but since the second light is emitted from a position different from that of the first light, it is necessary to use a multiplexing member Cm. As the multiplexing member Cm in FIG. 10, a member that transmits the first light and reflects the second light is used. The first light and the second light via the lens Lg are incident into the light guide 29 as an incidence ray Eb.

The multiplexing member Cm is a dichroic mirror, a dichroic prism, or the like, and can transmit or reflect light according to a wavelength to multiplex the light to be incident into the light guide 29. The wavelength of the light reflected or transmitted by the multiplexing member Cm can be set to any value. The multiplexing member Cm need only be provided on an optical path of the light transmitted through the beam splitter S, that is, on a side opposite to each light source and the lens L across the beam splitter S, and the installation position of the multiplexing member Cm is not particularly limited as long as multiplexing can be performed.

As shown in FIG. 11, the light source unit 20 including the two different light sources 25a and 25b and the photodetectors 26a and 26b corresponding to the respective light sources receives a drive current from the amount-of-light controller 24. Each light source emits light according to a drive current value, and each photodetector transmits information on the detected amount of received light to the amount-of-light controller 24 and the measurement unit 22.

In the single-frame observation mode, the first light source 25a and the second light source 25b are simultaneously turned on, or only one of the first light source 25a and the second light source 25b is turned on to emit light to capture a normal image that is illuminated with white light. In a case in which only one of the first light source 25a and the second light source 25b is turned on, broadband light such as white light is preferable, and in a case in which both the first light source 25a and the second light source 25b are turned on, it is preferable that two types of light emission are combined to make broadband light such as white light.

In the multi-frame observation mode, multi-frame light emission is performed in which the first illumination light and the second illumination light are switched to be turned on and off alternately or in a fixed pattern. One of the first illumination light and the second illumination light may be broadband light and the other may be special light, or a combination of the first illumination light and the second illumination light may be used to obtain broadband light. Wavelength ranges of the first illumination light and the second illumination light may be different from each other or may be the same as each other.

Even in a case in which the target integrated amount of light IL is obtained within the global exposure period T0, in a case in which the amount of light emission is too low, it may take a long time to perform switching from a turning-off state to a turning-on state, or a ratio of the fluctuation width of the variation to the amount of light emission may become large. Therefore, it is preferable to set the lowest value for light emission with a low amount of emission and to maintain the light emission at a certain amount of light emission or more.

As shown in FIG. 12, in light emission in which the first illumination light and the second illumination light are switched to be turned on and off alternately, for example, there is a pattern in which the first illumination light with the global exposure period T0 of 10 ms, a target amount of light emission of 100, and an exposure period of 10 ms, and the second illumination light with a target amount of light emission of 40 and an exposure period of 5 ms are alternately emitted. A vertical axis represents a first amount of light emission and a second amount of light emission, which are the amounts of light emission of the first illumination light and the second illumination light, and a horizontal axis represents a time. In a case in which illumination light is switched every frame, the first illumination light is emitted in an N-th frame, the second illumination light is emitted in an (N+1)th frame, the first illumination light is emitted in an (N+2)th frame as in the N-th frame, and the second illumination light is emitted in an (N+3)th frame as in the (N+1)th frame. The target amount of light emission is an amount of light emission in a case in which the illumination light is emitted according to the target drive current value in each frame. The integrated amount of light, which is a total value of the cumulative amounts of light, is a range surrounded by diagonal lines in each frame.

With respect to this, in order to reliably acquire the target integrated amount of light within the global exposure period even though there is a variation in the drive current, light is emitted in a light emission pattern in which the target amount of light emission of each illumination light is increased. The amount of light emission is a relative numerical value in the emission of each illumination light, and the unit thereof is not particularly limited. The variation in the amount of light is not displayed.

As shown in FIGS. 13 to 17, five patterns of increasing the first amount of light emission and the second amount of light emission in the multi-frame observation mode will be described as first to fifth patterns. In addition, as in FIG. 12, the variation in the amount of light is not displayed in FIGS. 13 to 17. The amount of light emission in a case in which light is emitted according to the target drive current value shown in FIG. 12 is indicated by a dotted line.

As shown in FIG. 13, in the first pattern, the amount of light emission is increased from a normal value by a constant magnification. For example, by increasing the target amount of light emission by 10%, the first amount of light emission is set to 110 and the second amount of light emission is set to 44.

As shown in FIG. 14, in the second pattern, the amount of light emission is increased from a normal value by a fixed value. For example, by increasing the amount of light, the first amount of light emission is set to 105 and the second amount of light emission is set to 45.

As shown in FIG. 15, in the third pattern, a rate of increase in the amount of light is changed according to the amount of light emission to increase the amount of light emission from the normal value. Since the imaging variation increases as the amount of light is low, a rate of increase for light emission in which the normal value of the amount of light is low is set high. For example, the first amount of light emission is set to 110 and the second amount of light emission is set to 48 by increasing the amount of light by 20% in a case in which the amount of light is 50 or less and increasing the amount of light by 10% in a case in which the amount of light is 51 or more.

As shown in FIG. 16, in the fourth pattern, the amount of light emission is set to a fixed value in a case in which the target amount of light emission is equal to or smaller than a certain value, and the amount of light emission is increased by a certain magnification from the normal value in a case in which the target amount of light emission is a value larger than the certain value. For example, the first amount of light emission is set to 110 and the second amount of light emission is set to 55 by fixing the amount of light emission to 55 in a case in which the target amount of light emission is 50 or less and increasing the amount of light emission by 10% in a case in which the target amount of light emission is 51 or more.

As shown in FIG. 17, in the fifth pattern, light is emitted in accordance with the end of the global exposure period T0, unlike the first to fourth patterns. A method of increasing the amount of light emission may be any method of the first to fourth patterns. In FIG. 17, an example in which the amount of light emission is increased by 10 as the fixed value in the second pattern is used. The light emission timing is adjusted such that the light emission ends at a timing slightly before than the end of the global exposure period T0, taking into account the influence of the variation in the drive current.

In any light emission pattern, the observation target is illuminated with illumination light of each frame, an image is captured by the imaging sensor 44 to acquire a captured image, and one observation image is generated using a plurality of captured images having different illumination light beams. In the multi-frame observation mode, one observation image can be generated from captured images of frames in which different illumination light beams are consecutively emitted, for example, a captured image of an N-th frame and a captured image of an (N+1)th frame. Another method may be used as a method of generating the observation image from the plurality of captured images.

A series of flow of an operation of controlling the amount of light during strobe light emission in the endoscope system 10 according to the present embodiment will be described with reference to a flowchart shown in FIG. 18. The endoscope system 10 sets the observation mode to the multi-frame observation mode by a user operation with respect to any one of the endoscope 11, the processor device 13, or the light source device 12 (step ST110). In a case in which the mode is switched to the multi-frame observation mode, information on a target amount of light emission of each frame included in the light emission pattern in the multi-frame observation mode is acquired (step ST120). Based on the information on the target amount of light emission such as a target drive current value, a drive current value is determined and light emission with the amount of light emission larger than the target amount of light emission is started, with the same illumination light as that in the light emission pattern in the multi-frame observation mode (step ST130). In regard to determination of the light emission pattern, the first to fifth patterns and the like may be registered in advance, and may be changed by an user operation or the like in a case in which there is a change from the registered contents, or may be set each time the light emission is started.

In a case in which the light emission in the multi-frame observation mode is started, the photodetector 26 receives the illumination light of the corresponding light source 25 to perform detection (step ST140). The measurement unit 22 acquires the amount of received light from the photodetector 26 for each sampling period a plurality of times within the light emission period, and calculates the amount of light emitted from the light source, from the amount of received light (step ST150). In addition, the measurement unit 22 acquires a cumulative amount of light obtained by accumulating the amount of light emission for each sampling period within the light emission period (step ST160). The light emission is stopped by the amount-of-light controller 24 in a case in which the integrated amount of light, which is a total value of the cumulative amounts of light, reaches the target integrated amount of light IL (step ST170). In a case in which light is emitted from the same light source consecutively in the next frame (Y in step ST180), the same light emission is repeated (step ST130).

In the next frame, in a case in which light is not emitted from the same light source (N in step ST180) or in a case in which switching is made to a different light source (Y in step ST190), a target amount of light emission in the switched light source is acquired, and light is emitted with an amount of light emission larger than the target amount of light emission is acquired (step ST120). In the next frame, in a case in which light is not emitted from the same light source (N in step ST180) and in a case in which switching is not made to a different light source (N in step ST190), the mode is switched from the multi-frame observation mode to the single-frame observation mode, and the series of flow ends (step ST200). The light emission period is an exposure period during which imaging is performed, and one observation image is generated from the plurality of captured images acquired for each light emission period. The endoscopic observation may be ended as it is without switching to the single-frame observation mode.

The multi-frame observation mode in the above flowchart has a content in which light emission is performed in each frame, in which a non-emission frame is not included, and each frame has a light emission pattern in which either the first illumination light or the second illumination light is emitted, but the non-emission frame may be included in the light emission pattern in the multi-frame observation mode.

Although the light source device 12 comprising two light sources that emit two types of illumination light beams has been described as an example, the light source device 12 that emits more types of illumination light beams may be used. For example, the multi-frame observation mode may be executed in the light source device 12 comprising four types of light sources that emit light beams having wavelength ranges different from each other. Hereinafter, a light source device using four types of light sources will be described.

As shown in FIG. 19, the light source device 12 comprising four types of light sources comprises: the first light source 25a, the second light source 25b, a third light source 25c, and a fourth light source 25d; and the first photodetector 26a, the second photodetector 26b, a third photodetector 26c, and a fourth photodetector 26d corresponding to the respective light sources. In the light source unit 20, as in FIG. 3, each photodetector receives light from the corresponding light source, which is partially reflected by the beam splitter S, and the light not reflected from the corresponding light source becomes an incidence ray Ec that is incident into the light guide 29 via the lens Lg. The second light to the fourth light are incident into the light guide 29 via the lens Lg as in the case of the first light, but are emitted from positions different from that of the first light. Therefore, it is necessary to make the light incident into the light guide 29 using the multiplexing member Cm. The transmission and reflection bandwidths of each of a plurality of the multiplexing members Cm need only be a bandwidth in which the second light to the fourth light are incident into the light guide 29.

As shown in FIG. 20, in the light source unit 20, the first light source 25a to the fourth light source 25d each independently receive a drive current for controlling the amount of light and the turning-off timing from the amount-of-light controller 24. Each of the light sources 25a to 25d emits light according to a drive current value, and each of the photodetectors 26a to 26d transmits information on the detected amount of received light to the amount-of-light controller 24 and the measurement unit 22. In order to determine the turning-off timing, the amount of received light of the reflected light of the light emitted from each light source, which is detected by each photodetector, is used.

As shown in FIG. 21, as examples of four types of light sources that emit light beams having wavelengths different from each other, the light source device 12 comprising the first light source 25a that emits red light R as the first light, the second light source 25b that emits green light G as the second light, the third light source 25c that emits blue light B as the third light, and the fourth light source 25d that emits violet light V as the fourth light is used.

In the single-frame observation mode, the respective light sources are simultaneously turned on to simultaneously emit the first light to the fourth light and capture a normal image that is illuminated with white light. In the multi-frame observation mode, multi-frame light emission is performed in which light emission with the first light to the fourth light is switched to be turned on and off in at least two or more types of light emission patterns. As the light emission pattern, frames in which the first light to the third light are simultaneously emitted and a frame in which only the fourth light is emitted are alternately repeated to create a display image. Since the fourth light is emitted in a different frame, an observation image in which only the fourth light is enhanced can be obtained.

The present invention is not limited to the light source device 12 comprising four types of light sources that emit light beams having wavelengths different from each other, and three types or five or more types of light sources may be used. In addition, it is preferable that the range of the wavelength of light to be emitted is appropriately changed depending on the use application. For example, a light source that emits short-wavelength blue light and a light source that emits long-wavelength blue light may be used instead of the blue light B.

In the above embodiment, the hardware structure of a processing unit that executes various kinds of processing, such as the measurement unit 22, the amount-of-light controller 24, the imaging controller 45, the image signal acquisition unit 50, the DSP 51, the noise reduction unit 52, the image processing unit 53, and the display controller 56, is various processors as shown below. The various processors include a central processing unit (CPU) that is a general-purpose processor that executes software (programs) to function as various processing units, a graphical processing unit (GPU), a programmable logic device (PLD) that is a processor capable of changing a circuit configuration after manufacture, such as a field programmable gate array (FPGA), and an exclusive electric circuit that is a processor having a circuit configuration exclusively designed to execute various kinds of processing.

One processing unit may be configured of one of these various processors, or may be configured of a combination of two or more processors of the same type or different types (for example, a plurality of FPGAs, a combination of a CPU and an FPGA, or a combination of a CPU and a GPU). In addition, a plurality of processing units may be configured of one processor. As an example in which the plurality of processing units are configured of one processor, first, as typified by computers such as a client or a server, one processor is configured of a combination of one or more CPUs and software, and this processor functions as the plurality of processing units. Second, as typified by a system on chip (SoC) or the like, a processor that realizes the functions of the entire system including the plurality of processing units by using one integrated circuit (IC) chip is used. As described above, the various processing units are configured using one or more of the various processors as a hardware structure.

Furthermore, the hardware structure of the various processors is more specifically an electric circuit (circuitry) having a form in which circuit elements such as semiconductor elements are combined. In addition, a hardware structure of a storage unit is a storage device such as a hard disc drive (HDD) or a solid state drive (SSD).

### EXPLANATION OF REFERENCES

10: endoscope system
11: endoscope
11a: insertion part
11b: operation part
11c: bendable part
11d: distal end part
11e: mode selector switch
12: light source device
13: processor device
14: display
15: user interface
20: light source unit
22: measurement unit
24: amount-of-light controller
25: light source
25a: first light source
25b: second light source
25c: third light source
25d: fourth light source
26: photodetector
26a: first photodetector
26b: second photodetector
26c: third photodetector
26d: fourth photodetector
29: light guide
30: illumination optical system
32: illumination lens
40: imaging optical system
42: objective lens
44: imaging sensor
45: imaging controller
46: CDS/AGC circuit
48: A/D converter
50: image signal acquisition unit
51: DSP
52: noise reduction unit
53: image processing unit
56: display controller
AL1: cumulative amount of light
AL2: cumulative amount of light
AL3: cumulative amount of light
ALn: cumulative amount of light
B: blue light
Cm: multiplexing member
DFX: definition line
DFY: definition line
Ea: incidence ray
Eb: incidence ray
Ec: incidence ray
G: green light
IL: target integrated amount of light
ILa: integrated amount of light
L: lens
Lg: lens
R: red light
S: beam splitter
ST110 to ST200: step
V: violet light

What is claimed is:

1. An endoscope system comprising:
at least one light source having a semiconductor light emitting element;
a photodetector that receives a part of light from the light source and detects an amount of received light; and
one or more processors configured to:
determine a drive current value that is a value larger than a target drive current value of the light source set in advance;
cause the light source to emit illumination light with an amount of light emission corresponding to the drive current value in a light emission period of the light source that is shorter than a determined exposure period;
acquire the amount of received light from the photodetector for each certain period a plurality of times within the light emission period;
calculate a cumulative amount of light that is the amount of light emission for each certain period, based on the amount of received light;
turn off the illumination light for a predetermined period in a case in which a total value of the cumulative amounts of light reaches a target integrated amount of light set in advance;
acquire a captured image obtained by imaging an observation target during the light emission period; and
generate one observation image from a plurality of the captured images acquired for each light emission period.

2. The endoscope system according to claim 1,
wherein the one or more processors are configured to cause the illumination light to be emitted at the drive current value increased from the target drive current value by a constant magnification.

3. The endoscope system according to claim 1,
wherein the one or more processors are configured to cause the illumination light to be emitted at the drive current value increased from the target drive current value by a fixed value.

4. The endoscope system according to claim 1,
wherein the one or more processors are configured to cause the illumination light to be emitted at the drive current value that is increased by a first magnification in a case in which the target drive current value is equal to or larger than a threshold value and is increased by a second magnification higher than the first magnification in a case in which the target drive current value is less than the threshold value.

5. The endoscope system according to claim 1,
wherein the one or more processors are configured to cause the illumination light to be emitted at the drive current value that is increased by a constant magnification in a case in which the target drive current value is equal to or larger than a threshold value and is increased by a fixed value in a case in which the target drive current value is less than the threshold value.

6. The endoscope system according to claim 1,
wherein the one or more processors are configured to set a light emission timing of the illumination light in accordance with an end of the determined exposure period.

7. The endoscope system according to claim 1,
wherein the certain period is 0.5 μs or more and 100 μs or less.

8. The endoscope system according to claim 1,
wherein the one or more processors are configured to:
acquire a variation between a set value of the target drive current value and an actual measurement value of a drive current transmitted to the light source; and
sets a lower limit value of the drive current value based on the variation.

9. The endoscope system according to claim 8,
wherein the one or more processors are configured to determine a method of increasing the drive current value with respect to the target drive current value according to the lower limit value.

10. The endoscope system according to claim 1,
wherein the one or more processors are configured to control exposure of light incident into an imaging sensor through a shutter control in a state where the light source continues to emit light, instead of light emission and turning-off of the light source.

11. The endoscope system according to claim 1,
wherein the semiconductor light emitting element is a laser diode or a light emitting diode.

12. The endoscope system according to claim 1,
wherein the one or more processors are configured to cause first illumination light and second illumination light, which are light beams having different wavelengths, to be emitted according to the respective drive current values.

13. A method of operating an endoscope system including at least one light source having a semiconductor light emitting element and a photodetector that receives a part of light from the light source and detects an amount of received light, the method comprising:
a step of determining a drive current value that is a value larger than a target drive current value of the light source set in advance;
a step of causing the light source to emit illumination light with an amount of light emission corresponding to the drive current value in a light emission period of the light source that is shorter than a determined exposure period;
a step of acquiring the amount of received light from the photodetector for each certain period a plurality of times within the light emission period;
a step of calculating a cumulative amount of light that is the amount of light emission for each certain period, based on the amount of received light;
a step of turning off the illumination light for a predetermined period in a case in which a total value of the cumulative amounts of light reaches a target integrated amount of light set in advance;
a step of acquiring a captured image obtained by imaging an observation target during the light emission period; and
a step of generating one observation image from a plurality of the captured images acquired for each light emission period.
